(19) United States
(12) Patent Application Publication
(10) Pub. No.: US 2022/0380765 A1
(43) Pub. Date: Dec. 1, 2022

(54) TARGETING NONSENSE-MEDIATED DECAY TO ACTIVATE P53 PATHWAY FOR THE TREATMENT OF CANCER

(71) Applicant: Board of Regents, The University of Texas System, Austin, TX (US)

(72) Inventors: John V. HEYMACH, Houston, TX (US); Jayanthi GUDIKOTE, Houston, TX (US); Tina CASCONE, Houston, TX (US)

(73) Assignee: Board of Regents, The University of Texas System, Austin, TX (US)

(21) Appl. No.: 17/773,682

(22) PCT Filed: Nov. 2, 2020
(86) PCT No.: PCT/US2020/058548

§ 371 Date: May 2, 2022

Related U.S. Application Data
(60) Provisional application No. 62/929,840, filed on Nov. 2, 2019.

(51) Int. Cl. C12N 15/113; A61K 31/706; A61K 31/522; A61K 31/704; A61K 31/365; A61K 31/12; A61K 31/7036; A61K 31/366; A61K 31/4745; A61K 31/7048; A61K 31/235; A61K 31/155; A61K 31/496; A61K 31/4545; A61K 31/428; A61K 31/122; A61P 35/00; C12Q 1/6886

(52) U.S. Cl. CPC C12N 15/113; A61K 31/706; A61K 31/522; A61K 31/704; A61K 31/365; A61K 31/12; A61K 31/7036; A61K 31/366; A61K 31/4745; A61K 31/7048; A61K 31/235; A61K 31/155; A61K 31/496; A61K 31/4545; A61K 31/428; A61K 31/122; A61P 35/00; C12Q 1/6886; C12N 2310/11; C12N 2310/14; C12Q 2600/106; C12Q 2600/156

(57) ABSTRACT

The present disclosure provides methods of treating cancer in a patient determined to have a p53 mutation by administering an inhibitor of nonsense mediated decay. The patient may be further administered an inhibitor of mRNA splicing and/or an inhibitor of MDM.

This application claims the benefit of U.S. Provisional Patent Application Ser. No. 62/929,840, filed Nov. 2, 2019, the entirety of which is incorporated herein by reference.

This invention was made with government support under grant number CA070907 awarded by the National Institutes of Health. The government has certain rights in the invention.

### BACKGROUND

### 1. Field

The present invention relates generally to the field of molecular biology and medicine. More particularly, it concerns methods of treating patients with p53-deficient cancers.

### 2. Description of Related Art

Loss of p53 function is the major cause of malignancy in many types of cancers. Truncating mutations in TP53 gene, amplification/overexpression of the negative regulator MDM2 leading to MDM2-mediated p53 protein degradation and HPV-E6-mediated degradation of p53 are the foremost reasons for its deficiency. Currently, therapeutic strategies to reactivate p53 and overcome p53 deficiency are lacking.

### SUMMARY

Embodiments of the present disclosure provides methods and compositions for treating p53-deficient cancer in a subject comprising administering a therapeutically effective amount of an inhibitor of nonsense-mediated decay (NMD) to said subject. In a first embodiment, there is provided a method of treating a subject determined to have a p53-deficient cancer, the method comprising restoring p53 function in the subject's tumor by administering a therapeutically effective amount of an inhibitor of nonsense-mediated decay (NMD) to said subject.

In some aspects, the p53-deficient cancer comprises a C-terminal p53 mutation. In particular aspects, the C-terminal p53 mutation is a nonsense or missense mutation downstream of exon 9. In specific aspects, the p53-deficient cancer comprise a mutant p53 with premature termination codons (PTCs) in the coding region. For example, the PTCs are at residue 331, 342, or 349. In some aspects, the p53-deficient cancer comprises the p53α, p53β, or p53γ isoform.

In certain aspects, the p53-deficient cancer is a MDM2 amplified or overexpressing cancer. In some aspects, the p53-deficient cancer is a MDMX amplified or overexpressing cancer. In particular aspects, the p53-deficient cancer is an HPV-positive cancer.

In some aspects, the subject was determined to have a p53 mutation by analyzing a genomic sample from said subject. In particular aspects, the genomic sample is isolated from saliva, blood, urine, normal tissue, or tumor tissue. In certain aspects, the presence of a p53 mutation is determined by nucleic acid sequencing or PCR analyses.

In some aspects, the inhibitor of NMD is or comprises 5-azacytidine, NMDI-14 (530838), NMDI-1, Caffiene, Wortmannin, Amlexanox, 5-Azacytidine, Cycloheximide, Pateamine A (PatA), Emetine, Digitoxin, Digoxin, Proscillaridin, Lanatoside C, Ouabain, Docusate sodium, butyl paraben, ethacridine lactate, Thapsigargin, Calcium ionophore A23187, Curcumin, Geneticin, Gentamicin, Negamycin, RTC #13, RTC #14, G-418, PTC124 (Ataluren), Tylosin, Compound C, ouabain, digoxin, doxorubicin, SMG-1 inhibitors (e.g., IACS 14140), UPF1 antisense RNA or UPF1 siRNA. In certain aspects, the inhibitor of NMD comprises an antisense oligonucleotide molecule, peptides, proteins, nucleic acids, organic or inorganic molecules which inhibit the nonsense mediated decay pathway, or molecules that inhibit function and/or expression of at least one factor associated with the NMD pathway (e.g. RENT1, RENT2, eIF4A, UPF1, UPF2, UPF3B, RNPS1, Y14, MAGOH, NMD1, SMG, or combinations thereof). In particular aspects, the amount of the NMD inhibitor is sufficient to partially restore p53 activity in cells of the cancer. In specific aspects, the method further comprises determining whether p53 activity is restored by measuring an increased mRNA expression level of p53 transcriptional targets. For example, the p53 targets include, but are not limited to MDM2, MDMX, GADD45A, BAX, GDF15, SUSD6 (TMPS, DRAGO, KIAA0247), PLK3, BTG2, TIGAR (C12orf5), TNFRSF10B, PPM1D, AEN, PLK2, SESN1, FAS, KITLG GADD45A, p21 and/or PUMA.

In additional aspects, the method further comprises administering a therapeutically effective amount of an inhibitor of mRNA splicing. In certain aspects, the inhibitor of mRNA splicing is a serine/arginine-rich splicing factor (SRFS) inhibitor. In certain aspects, the inhibitor of mRNA splicing is a serine/arginine-rich splicing factor 1 (SRFS1, ASF1/SF2) inhibitor. For example, SRFS inhibitors include, without limitation, TG003, diospyrin, a SF3B1 inhibitor, a thailanstatin, chlorohexidine, SPHINX 31, or SRPIN 340. In further aspects, the mRNA splicing inhibitor comprises SF3B1 inhibitors, thailanstatins, or any small molecules described to directly alter splicing and/or post-translational modifications of spliceosomal proteins.

In further aspects, the method further comprises administering an additional anti-cancer therapy. In some aspects, the additional anti-cancer therapy is chemotherapy, radiotherapy, gene therapy, surgery, hormonal therapy, anti-angiogenic therapy or immunotherapy. In particular aspects, the additional anti-cancer therapy is radiotherapy, chemotherapy, radiation, immunotherapy, or a combination thereof. In specific aspects, the additional anti-cancer therapy is an MDM2 targeting drug. For example, the MDM2 targeting drug is Nutlin-3, RG7112 (RO5045337), RG7388 (RO5503781, Idasanutlin), SAR405838 (MI-77301), MK-8242 (SCH 900242), DS-3032b, HDM201, CGM097, ALRN-6924 or AMG-232.

In some aspects, the NMD inhibitor and/or additional anti-cancer therapy are administered intravenously, subcutaneously, intraosseously, orally, transdermally, in sustained release, in controlled release, in delayed release, as a suppository, or sublingually. In certain aspects, administering the NMD inhibitor and/or anti-cancer therapy comprises local, regional or systemic administration. In some aspects, the NMD inhibitor and/or anti-cancer therapy are administered two or more times.

In particular aspects, the subject is human. In some aspects, the cancer is sarcoma, breast cancer, lung cancer, or glioblastoma. For example, the lung cancer is non-small cell lung cancer or a small cell lung cancer. In some aspects, the HPV-positive cancer is head and neck squamous cell carcinoma, oropharyngeal cancer, cervical cancer, anal cancer, penile cancer, or vulvar cancer.

A further embodiment provides a pharmaceutical composition comprising an NMD inhibitor and mRNA splicing inhibitor. In some aspects, the NMD inhibitor is 5-azacytidine, NMDI-14 (530838), NMDI-1, Caffiene, Wortmannin, Amlexanox, 5-Azacytidine, Cycloheximide, Pateamine A (PatA), Emetine, Digitoxin, Digoxin, Proscillaridin, Lanatoside C, Ouabain, Docusate sodium, butyl paraben, ethacridine lactate, Thapsigargin, Calcium ionophore A23187, Curcumin, Geneticin, Gentamicin, Negamycin, RTC #13, RTC #14, G-418, PTC124 (Ataluren), Tylosin, Compound C, ouabain, digoxin, doxorubicin, SMG-1 inhibitors (e.g., IACS 14140), UPF1 antisense RNA or UPF1 siRNA. In certain aspects, the inhibitor of NMD comprises an antisense oligonucleotide molecule, peptides, proteins, nucleic acids, organic or inorganic molecules which inhibit the nonsense mediated decay pathway, or molecules that inhibit function and/or expression of at least one factor associated with the NMD pathway (e.g. RENT1, RENT2, eIF4A, UPF1, UPF2, UPF3B, RNPS1, Y14, MAGOH, NMD1, SMG, or combinations thereof). In certain aspects, the mRNA splicing inhibitor is TG003, diospyrin, a SF3B1 inhibitor, a thailanstatin, chlorohexidine, SPHINX 31, or SRPIN 340.

Another embodiment provides a composition comprising an effective amount of an NMD inhibitor and mRNA splicing inhibitor for use in the treatment of a p53-deficient cancer. Also provided herein is the use of composition of comprising an effective amount of an NMD inhibitor and mRNA splicing inhibitor for the treatment of a p53-deficient cancer in a subject. In some aspects, the NMD inhibitor is 5-azacytidine, NMDI-14 (530838), NMDI-1, Caffiene, Wortmannin, Amlexanox, 5-Azacytidine, Cycloheximide, Pateamine A (PatA), Emetine, Digitoxin, Digoxin, Proscillaridin, Lanatoside C, Ouabain, Docusate sodium, butyl paraben, ethacridine lactate, Thapsigargin, Calcium ionophore A23187, Curcumin, Geneticin, Gentamicin, Negamycin, RTC #13, RTC #14, G-418, PTC124 (Ataluren), Tylosin, Compound C, ouabain, digoxin, doxorubicin, SMG-1 inhibitors (e.g., IACS 14140), UPF1 antisense RNA or UPF1 siRNA. In certain aspects, the inhibitor of NMD comprises an antisense oligonucleotide molecule, peptides, proteins, nucleic acids, organic or inorganic molecules which inhibit the nonsense mediated decay pathway, or molecules that inhibit function and/or expression of at least one factor associated with the NMD pathway (e.g. RENT1, RENT2, eIF4A, UPF1, UPF2, UPF3B, RNPS1, Y14, MAGOH, NMD1, SMG, or combinations thereof).

In yet another embodiment, there is provided a method of identifying a subject who is a candidate for an NMD inhibitor therapy comprising analyzing the sequence of a P53 gene from a cancer in the subject having been determined to have a p53-deficient cancer. In some aspects, the sequence is analyzed from a genomic sample from said subject. In certain aspects, the genomic sample is isolated from saliva, blood, urine, normal tissue, or tumor tissue. In particular aspects, analyzing comprises performing nucleic acid sequencing or PCR analyses. In some aspects, a subject determined to have a p53 nonsense mutation downstream of exon 9 and/or PTCs in the p53 coding region is a candidate for an NMD inhibitor therapy. For example, the PTCs are at residue 331, 342, or 349. In some aspects, a subject determined to have a p53β isoform or p53γ isoform is a candidate for an NMD inhibitor therapy.

In additional aspects, the method further comprises administering an NMD inhibitor therapy to said subject identified to be a candidate. In some aspects, the NMD inhibitor therapy comprises administering 5-azacytidine, wortmannin, IACS 14140, Ataluren (Translarna), Amlexanox, NMDI-1, UPF1 antisense RNA, or UPF1 siRNA.

In further aspects, the method further comprises administering a therapeutically effective amount of an inhibitor of mRNA splicing. In some aspects, the inhibitor of mRNA splicing is a serine/arginine-rich splicing factor (SRFS) inhibitor. In some aspects, the inhibitor of mRNA splicing is a serine/arginine-rich splicing factor 1 (SRFS1, ASF1/SF2) inhibitor. In certain aspects, the SRFS inhibitor is TG003, diospyrin, a SF3B1 inhibitor, a thailanstatin, chlorohexidine, SPHINX 31, or SRPIN 340.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1M: NMD inhibition induces p53β/γ expression and stimulates p53 pathway in cancer cells overexpressing negative regulators of p53. a, Schematic showing functional domains (black lines) in p53 protein. aa, amino acids spanning each domain. b, Schematic representation of p53α and β/γ isoforms generated by alternative splicing of intron-9. C-terminal MDM2 binding region depicted in black. Stop signs in exons i9 indicate PTCs acquired by alternative splicing. c, d, mRNA expression FC of p53β and p53γ respectively, upon NMDi treatment (1 μM) in NSCLC and GBM cell lines. e, Western blots showing truncated p53 induction in NMDi treated NSCLC and GBM cell lines. f, g, mRNA expression FC of p53β and p53γ respectively, upon NMDi treatment in HPV⁺HNSCC cell lines. h, Western analysis showing truncated p53 induction in NMDi treated HPV⁺HNSCC cell lines. j, k, mRNA expression FC of GADD45A, p21 and PUMA respectively upon NMDi treatment in cell lines shown. 1, Western analysis showing p53 pathway activation in NMDi treated cells. SE, short exposure, LE, long exposure. m, Quantification of the western blots shown in 1 (2 technical repeats). RT-qPCR analysis shown (n=2 technical repeats) are representatives of 3 independent experiments. Mean±s.e., p values, two tailed t-tests, *≤0.05, **<0.01, ***<0.001, ****<0.0001.

FIGS. 2A-2I: NMD inhibitor rescues p53 expression and promotes p53 pathway activation in p53 mutant cell lines. a, Schematic representation of mutant p53. b, mRNA expression fold change (FC) of total p53 in either control or NMDi treated cells harboring nonsense mutations at codon 331 (H2228), 342 (TCCSUP) or 349 (UACC-893). c, Western blots showing increased expression of truncated p53 protein upon NMDi treatment. d, e, mRNA expression FC of p53 and p53γ isoforms in control and NMDi treated cells. f, g, h, mRNA expression FC of p53 transcriptional targets GADD45A, p21 and PUMA in control and NMDi treated cells. i, Western blots showing increased p21 and PUMA protein expression upon NMD inhibition. mRNA expression assessed by RT-qPCR. Data shown (n=2 technical replicates) are representatives of 3 independent experiments. Mean±s.e., p values, two tailed t-tests, *≤0.05, **<0.01, ***<0.001, ****<0.0001. GAPDH (H2228) or vinculin (HTB-5 and CRL-1902) were used as loading controls for Western blots.

FIGS. 3A-3E: NMD inhibition reduces colony forming ability and tumor growth. a, b, c, Clonogenic assay showing reduction in colony forming ability upon NMD inhibition. d, Subcutaneous xenograft tumor growth in nude mice using A549. e, Orthotopic tumor growth model of UMSCC47 on nude mice tongue. Mice (n=10 per group) were treated either with vehicle or with the indicated doses of NMDi.

FIGS. 4A-4D: Model depicting the p53 restoration strategy in p53 deficient tumors. a, In normal cells, p53 pre-mRNA splicing results in p53α and p53β/γ mRNAs. p53β/γ mRNA is degraded by NMD, p53α mRNA is translated. b, NMD inhibition stabilizes p53β/γ mRNA generating p53β/γ protein. c, Combination of SR protein inactivation and NMD inhibition results in splicing shift and further increase in the expression of p53β/γ. d, In MDM2 amplified or HPV⁺ cells, p53α protein is degraded. Combining SR protein inactivation with NMD inhibition overcomes p53 deficiency by increasing the expression of p53β/γ that lack the C-terminal MDM2 binding sites. Stop sign in black, normal stop codon; stop sign in grey, PTC. p53 exons 1-9 are depicted in black, exons 10 and 11 are depicted in grey.

FIGS. 5A-5C: MDM2 copy number and mRNA expression status. a, mRNA expression and MDM2 copy number analysis of NSCLC cell lines, b, Western blot showing MDM2 expression in GBM cell lines. c, mRNA expression and MDM2 copy number analysis of HNSCC cell lines. Expression analysis done using RNAseq data. HPV status and TP53 mutation status are indicated. CN, copy number.

FIGS. 6A-6L: NMD inhibition activates p53 pathway in MDM2 overexpressing cell lines. a, p53β and p53γ mRNA expression FC upon NMDi treatment in H460 cell line. b, Western blot showing induction of p53β/γ protein expression upon NMD inhibition in H460. c, d, e, mRNA expression FC of GADD45A, p21, and PUMA respectively. f, Western analysis showing p53 pathway activation in NMDi treated A549 and GSC289. g, h, p53β and p53γ mRNA expression fold change (FC) respectively, assessed by RT-qPCR in cells with UPF1 depletion (siUPF1) or not (siControl). Mean±s.e. Data shown (n=2 technical replicates) is a representative of 2 or more independent transfection experiments. i, Western blot showing induction of truncated p53 protein upon UPF1 depletion. j, k, GADD45A and p21 mRNA expression FC respectively in cells depleted of UPF1 or not. 1, Western analysis showing increased p21 and puma expression in UPF1 depleted cells. RT-qPCR analysis shown (n=2 technical repeats) are representatives of 3 independent experiments. Mean±s.e., p values, two tailed t-tests, *≤0.05, **<0.01, ***<0.001, ****<0.0001.

FIGS. 7A-7G: p53γ contributes to the increased p53 pathway activation upon NMD inhibition in NSCLC cell line. a, p53β and p53γ mRNA expression FC upon NMDi treatment in A549 cells, treated with the siRNAs shown. b, Western analysis showing p53α (full-length) and truncated p53β/γ expression. c, d, e, mRNA expression FC of p53 transcriptional targets p21, GADD45A and PUMA respectively, upon NMDi treatment. f, g, Western analysis showing p21 and PUMA expression respectively upon NMDi treatment in A549 cells treated with siRNAs shown. Lower panels in f and g are the quantifications (2 technical repeats) of the Western blots, normalized to either GAPDH or Vinculin. RT-qPCR (n=2 technical repeats) and Western analysis shown are representatives of 3 independent experiments. Mean±s.e., p values, two tailed t-tests, *≤0.05, **<0.01, ***<0.001, ****<0.0001.

FIGS. 8A-8G: p53γ contributes to the increased p53 pathway activation upon NMD inhibition in HPV+ HNSCC cell line. a, p53β and p53γ mRNA expression FC upon NMDi treatment in HMS001 cells, treated with the siRNAs shown. b, Western analysis showing p53α (full-length) and truncated p53β/γ expression. c, d, e, mRNA expression FC of p53 transcriptional targets p21, GADD45A and PUMA respectively, upon NMDi treatment. f, g, Western analysis showing p21 and PUMA expression respectively upon NMDi treatment in A549 cells treated with siRNAs shown. Lower panels in f and g are the quantifications (2 technical repeats) of the Western blots, normalized to either GAPDH or Vinculin. RT-qPCR (n=2 technical repeats) and Western analysis shown are representatives of 3 independent experiments. Mean±s.e., p values, two tailed t-tests, *≤0.05, **<0.01, ***<0.001, ****<0.0001.

FIGS. 9A-9C: HNSCC and NSCLC cell lines are sensitive to NMDi. a, IC50 of NSCLC and HNSCC cell lines for NMDi treatment. b, HNSCC cells are more sensitive to NMDi than NSCLC cells. c, IC50 for HNSCC cell lines treated with the chemotherapy drugs shown and MDM2 inhibitor nutlin. IC50 shown are average from three technical replicates. Experiment was repeated two separate times.

FIGS. 10A-10B: NMD inhibition increases radiation sensitivity. Clonogenic survival assay of a, NSCLC and b, HNSCC cell lines treated with either NMDi or DMSO (control) and exposed to different doses of radiation.

FIGS. 11A-11B: NMDi treatment disrupts cell cycle progression. a, A549 and b, HMS001 cells were treated with either DMSO or NMDi (1 μM) for the indicated time points. Cells were fixed in 70% ethanol, stained with propidium iodide and analyzed by Flow Cytometry.

FIGS. 12A-12C: NMDi treatment reduces tumor growth and increases p53γ expression in tumors. a, Subcutaneous xenograft tumor growth in nude mice using UMSCC47. Mice (n=10 per group) were treated either with vehicle or with the indicated doses of NMDi. b,c, mRNA expression FC assessed by RT-qPCR in vehicle or NMDi treated tumor tissue samples derived from A549 (b) or UMSCC47 (c) subcutaneous xenografts. Mean±s.e., n=5 each.

FIGS. 13A-13E: Combining NMD inhibition with TG003 substantially increases the expression of p53β/γ and p53 transcriptional targets. a, b, p53α, p53β and p53γ mRNA expression FC assessed by RT-qPCR in A549 or UMSCC47 treated with either NMDi (1 μM) or TG003 (50 μM) or both. c, Western blot analysis showing increased expression of p53β/γ proteins in A549 and UMSCC47 cell lines treated with a combination of NMDi (1 μM) and TG003 (50 μM). d, e, mRNA expression FC of p53 transcriptional targets assessed by RT-qPCR in A549 or UMSCC47 treated as in a and b. RT-qPCR analysis shown are average of 3 independent experiments. Mean±s.e., p values, two tailed t-tests, *≤0.05, **<0.01, ***<0.001, ****<0.0001.

FIGS. 14A-14F: NSCLC and HNSCC cells are more sensitive to combination of NMDi and TG003. Relative cell viability of NSCLC (a,b,c) and HNSCC (d,e,f) cell lines treated with either NMDi or TG003 or a combination of both. Data shown are average from three technical replicates. Experiment was repeated two separate times.

### DESCRIPTION OF ILLUSTRATIVE EMBODIMENTS

TP53 is one of the highly mutated tumor suppressors in cancer. A vast majority of p53 mutations are truncating mutations that trigger mRNA degradation by nonsense-mediated decay (NMD), a regulator of aberrant mRNA stability. The present studies show that p53 and p53γ isoforms which retain the functional properties of canonical full-length p53 are NMD substrates. Inhibiting NMD rescues nonsense mutation-bearing p53 transcripts as well as p53 and p53γ isoforms. NMD inhibition leads to p53 pathway activation in both p53 mutant and non-mutant cancer cells and the relative contribution of p53γ is greater than that of p53 in promoting p53 pathway activation. The present studies further show that NMD inhibition increased radiosensitivity and inhibited tumor growth. Furthermore, dual inhibition of NMD and the splicing factor SFRS1 shifts p53 mRNA splicing from canonical p53α to the p53β/γ isoforms, markedly enhancing the effects of NMD inhibition. These results identify a novel therapeutic strategy for restoration of p53 function in p53 deficient cancers.

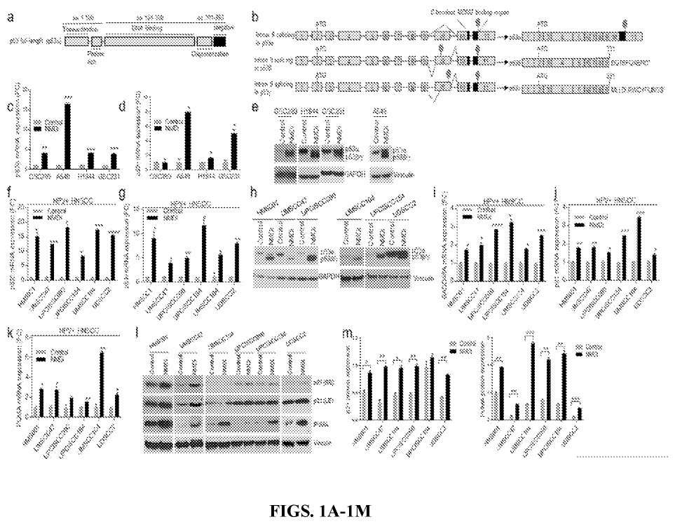

Specifically, the present studies explored whether p53 isoforms that are less susceptible to MDM2 and E6-mediated degradation can rescue p53 function. TP53 encodes twelve distinct isoforms generated by different promoters, alternative translation start sites or alternative splicing of intron 9. Among p53 isoforms, p53β and p53γ isoforms retain several key functions of wild-type p53. Moreover, unlike full-length p53α, these isoforms lack the C-terminal negative regulatory region containing MDM2 binding sites (lysine residues at positions 370, 372, 373, 381, 382 and 386) (FIG. 1A) and hence, are less susceptible for MDM2-mediated degradation. However, because these isoforms are generated by alternative splicing of intron-9 and bear premature termination codons (PTCs) in the coding region (FIG. 1B, exon i9), they are degraded by NMD, an RNA surveillance pathway that regulates the expression of aberrant mRNA transcripts including alternatively spliced mRNAs. It was shown that modulation of p53 intron 9 splicing to shift p53 mRNA splicing from a to β/γ isoforms in concomitance with NMD inhibition upregulates the p53β/γ isoforms and activates p53 pathway and this can be used as a therapeutic strategy to overcome p53 deficiency in MDM2 overexpressing and HPV-E6-driven tumors.

NMD was inhibited in several non-small cell lung cancer (NSCLC) cell lines and HPV-positive head and neck squamous cell carcinoma (HNSCC) cell lines either by RNAi-mediated depletion of the key regulator of NMD pathway, UPF1 or by using a pharmacological NMD inhibitor IACS 14140 that selectively inhibits another important regulator of NMD pathway, SMG1. NMD regulated transcripts were identified in NSCLC cell lines by performing whole transcriptomic analysis using RNAseq and quantitative real-time RT-PCR (qPCR) methods. Proteomic analysis of the downstream protein targets of NMD-regulated transcripts was done by reverse-phase protein array (RPPA) and western blotting.

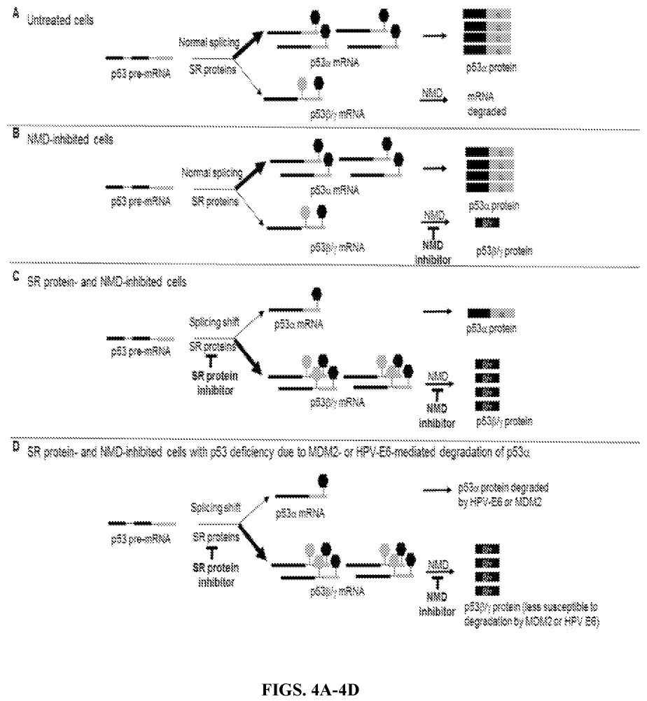

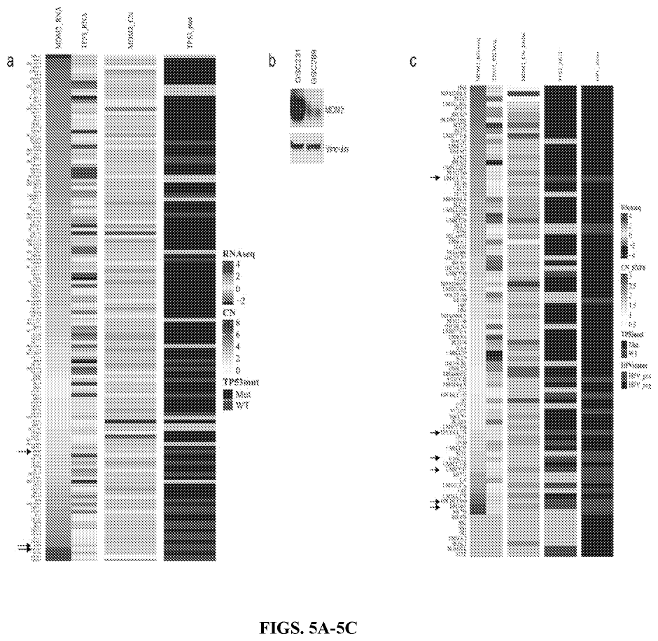

Accordingly, in certain embodiments, methods are provided herein for the use of NMD inhibition as a strategy to re-activate p53 and, in turn, impair tumor growth and sensitize tumor cells to commonly used therapeutic regimens. NMD inhibition can be used to stabilize p53β and γ isoforms and restore p53 activity in several major types of p53-deficient tumor cells, such as tumor cells with nonsense mutations downstream of exon 9, MDM2 overexpressing tumor cells, or HPV⁺ cells (e.g. HNSCC cells). Moreover, further methods are provided herein for the concomitant inhibition of NMD and mRNA splicing, such as inhibition of mRNA splicing factors (e.g., serine-arginine-rich splicing factors (SRSFs)) which alternative splicing of p53, to markedly increase the expression of p53β and p53γ isoforms (FIG. 4, 14). The methods may comprise inhibition of one or more members of the SRSF family, such as SRSF1, SRSF2, SRSF3, SRSF4, SRSF5, SRSF6, SRSF7, SRSF8, SRSF9, SRSF10, SRSF11, or SRSF12. Thus, the present methods can be used to effectively activate the p53 pathway, increase radiosensitivity, and reduce tumor growth by NMD inhibition, alone or in combination with agents targeting splicing factors, such as SRSFs (e.g., SFRS1), in cancers that are p53-deficient. P53-deficient cancers may have amplification or overexpression of MDM2 and MDMX (e.g., brain tumors, sarcomas, breast cancer, NSCLC, etc.) or are HPV positive cancers (e.g., oropharyngeal, cervical, anal, penile, and vulvar cancers). In these tumor types, most p53 isoforms (including the full length p53α isoform) are degraded in part through its interactions with MDM2, MDMX (for tumors overexpressing these factors) or HPV E6 protein (for HPV positive tumors). The inhibition of NMD reduces degradation of two alternatively spliced p53 isoforms, p53β and p53γ, which are functional p53 isoforms that are less sensitive to the degradation mediated by MDM2, MDMX, or HPV E6. The NMD inhibition and/or mRNA splicing inhibition may be alone or in combination with additional anti-cancer therapies, such as chemotherapy, radiation, immunotherapy, or some combination thereof (e.g. chemotherapy plus and NMD inhibitor; chemotherapy plus radiation plus NMD inhibitor; etc). NMD inhibitors could also be used in combination with other drugs targeting MDM2, such as Nutlin-3.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “about” means, in general, the stated value plus or minus 5%.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

“Hybridize” or “hybridization” refers to the binding between nucleic acids. The conditions for hybridization can be varied according to the sequence homology of the nucleic acids to be bound. Thus, if the sequence homology between the subject nucleic acids is high, stringent conditions are used. If the sequence homology is low, mild conditions are used. When the hybridization conditions are stringent, the hybridization specificity increases, and this increase of the hybridization specificity leads to a decrease in the yield of non-specific hybridization products. However, under mild hybridization conditions, the hybridization specificity decreases, and this decrease in the hybridization specificity leads to an increase in the yield of non-specific hybridization products.

A “probe” or “probes” refers to a polynucleotide that is at least eight (8) nucleotides in length and which forms a hybrid structure with a target sequence, due to complementarity of at least one sequence in the probe with a sequence in the target region. The polynucleotide can be composed of DNA and/or RNA. Probes in certain embodiments, are detectably labeled. Probes can vary significantly in size. Generally, probes are, for example, at least 8 to 15 nucleotides in length. Other probes are, for example, at least 20, 30 or 40 nucleotides long. Still other probes are somewhat longer, being at least, for example, 50, 60, 70, 80, or 90 nucleotides long. Probes can be of any specific length that falls within the foregoing ranges as well. Preferably, the probe does not contain a sequence complementary to the sequence(s) used to prime for a target sequence during the polymerase chain reaction.

“Oligonucleotide” or “polynucleotide” refers to a polymer of a single-stranded or double-stranded deoxyribonucleotide or ribonucleotide, which may be unmodified RNA or DNA or modified RNA or DNA.

A “modified ribonucleotide” or deoxyribonucleotide refer to molecules that can be used in place of naturally occurring bases in nucleic acid and includes, but is not limited to, modified purines and pyrimidines, minor bases, convertible nucleosides, structural analogs of purines and pyrimidines, labeled, derivatized and modified nucleosides and nucleotides, conjugated nucleosides and nucleotides, sequence modifiers, terminus modifiers, spacer modifiers, and nucleotides with backbone modifications, including, but not limited to, ribose-modified nucleotides, phosphoramidates, phosphorothioates, phosphonamidites, methyl phosphonates, methyl phosphoramidites, methyl phosphonamidites, 5′-β-cyanoethyl phosphoramidites, methylenephosphonates, phosphorodithioates, peptide nucleic acids, achiral and neutral internucleotidic linkages.

A “primer” or “primer sequence” refers to an oligonucleotide that hybridizes to a target nucleic acid sequence (for example, a DNA template to be amplified) to prime a nucleic acid synthesis reaction. The primer may be a DNA oligonucleotide, an RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acid under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions.

“Detection,” “detectable” and grammatical equivalents thereof refer to ways of determining the presence and/or quantity and/or identity of a target nucleic acid sequence. In some embodiments, detection occurs amplifying the target nucleic acid sequence. In other embodiments, sequencing of the target nucleic acid can be characterized as “detecting” the target nucleic acid. A label attached to the probe can include any of a variety of different labels known in the art that can be detected by, for example, chemical or physical means. Labels that can be attached to probes may include, for example, fluorescent and luminescence materials.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA) (TwistDx, Cambridg, UK), and self-sustained sequence replication (3 SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3^{rd} Edition).

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “nonsense-mediated decay (NMD)” as used herein refers to a mechanism that eliminates transcripts to regulate gene expression. For example, NMD can degrade mRNAs containing nonsense mutations, such as premature translation-termination codons (PTCs).

### II. P53 MUTATIONS

Certain embodiments of the present disclosure concern determining if a subject has one or more p53 mutations, such as p53 C-terminal mutations, including p53β isoform and p53γ isoform (FIG. 1B). The p53 isoforms, p53β and p53γ isoforms retain several key functions of wild-type p53. Moreover, unlike full-length p53α, these isoforms lack the C-terminal negative regulatory region containing MDM2 binding sites (lysine residues at positions 370, 372, 373, 381, 382 and 386). C-terminal mutations may be downstream of exon 9 on p53 and may introduce PTCs in the coding region, such as at residue 331, 342, or 349. Mutation detection methods are known the art including PCR analyses and nucleic acid sequencing as well as FISH and CGH. In particular aspects, the p53 mutations are detected by DNA sequencing, such as from a tumor or circulating free DNA from plasma.

The patient sample can be any bodily tissue or fluid that includes nucleic acids from the lung cancer in the subject. In certain embodiments, the sample will be a blood sample comprising circulating tumor cells or cell free DNA. In other embodiments, the sample can be a tissue, such as a lung tissue. The lung tissue can be from a tumor tissue and may be fresh frozen or formalin-fixed, paraffin-embedded (FFPE). In certain embodiments, a lung tumor FFPE sample is obtained.

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine, tumor, or expectorant. The sample itself will typically include nucleated cells (e.g., blood or buccal cells) or tissue removed from the subject including normal or tumor tissue. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of p53 mutations as described herein can be determined using methods known in the art. For example, gel electrophoresis, capillary electrophoresis, size exclusion chromatography, sequencing, and/or arrays can be used to detect the presence or absence of insertion mutations. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an insertion mutation as described herein. An insertion mutation can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular variant.

A set of probes typically refers to a set of primers, usually primer pairs, and/or detectably-labeled probes that are used to detect the target genetic variations used in the actionable treatment recommendations of the present disclosure. The primer pairs are used in an amplification reaction to define an amplicon that spans a region for a target genetic variation for each of the aforementioned genes. The set of amplicons are detected by a set of matched probes. In an exemplary embodiment, the present methods may use TaqMan™ (Roche Molecular Systems, Pleasanton, Calif.) assays that are used to detect a set of target genetic variations. In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing (Sears et al., 1992), solid-phase sequencing (Zimmerman et al., 1992), sequencing with mass spectrometry such as matrix-assisted laser desorption/ionization time-of-flight mass spectrometry (MALDI-TOF/MS; Fu et al., 1998), and sequencing by hybridization (Chee et al., 1996; Drmanac et al., 1993; Drmanac et al., 1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC, Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

In one example, a method of identifying a p53 mutation in a sample comprises contacting a nucleic acid from said sample with a nucleic acid probe that is capable of specifically hybridizing to nucleic acid encoding a mutated p53 protein, or fragment thereof incorporating a mutation, and detecting said hybridization. In a particular embodiment, said probe is detectably labeled such as with a radioisotope (³H, ³²P, or ³³P), a fluorescent agent (rhodamine, or fluorescein) or a chromogenic agent. In a particular embodiment, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, said probes of the present disclosure are provided in a kit for identifying p53 mutations in a sample, said kit comprising an oligonucleotide that specifically hybridizes to or adjacent to a site of mutation in the p53 gene. The kit may further comprise instructions for treating patients having tumors that contain p53 mutations with an NMD inhibitor based on the result of a hybridization test using the kit.

In another aspect, a method for detecting a p53 mutation in a sample comprises amplifying from said sample nucleic acids corresponding to exon 9 of said p53, or a fragment thereof suspected of containing a mutation, and comparing the electrophoretic mobility of the amplified nucleic acid to the electrophoretic mobility of corresponding wild-type p53 gene or fragment thereof. A difference in the mobility indicates the presence of a mutation in the amplified nucleic acid sequence. Electrophoretic mobility may be determined on polyacrylamide gel.

Alternatively, nucleic acids may be analyzed for detection of mutations using Enzymatic Mutation Detection (EMD) (Del Tito et al., 1998). EMD uses the bacteriophage resolvase T4 endonuclease VII, which scans along double-stranded DNA until it detects and cleaves structural distortions caused by base pair mismatches resulting from point mutations, insertions and deletions. Detection of two short fragments formed by resolvase cleavage, for example by gel electrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T4 endonuclease VII as demonstrated in U.S. Pat. No. 5,869,245.

### III. METHODS OF TREATMENT

Further provided herein are methods for treating or delaying progression of cancer in an individual comprising administering to the individual an effective amount of an NMD inhibitor, or a structurally similar inhibitor, to a subject determined have a p53-deficient cancer. In some aspects, the subject is further administered a mRNA splicing inhibitor.

The NMD inhibitor may be, but is not limited to, 5-azacytidine, wortmannin, IACS 14140, MilliporeSigma™ Calbiochem™ NMD Inhibitor, AMPK-selective inhibitor Compound C (Cheruiyot et al., 2018; incorporated herein by reference), tetracyclic compound NMDI1 (Gotham et al., 2015; incorporated herein by reference), VG1, Ataluren (Translarna), read-through compound (RTC)13, Amlexanox, synthetic aminoglycosides and nonaminoglycosides, cardiac glycosides, UPF1 antisense RNA, and UPF1 siRNA

In some aspects, the inhibitor of mRNA splicing is a SRSF inhibitor, such as a serine/arginine-rich splicing factor 1 (SRFS1, ASF1/SF2) inhibitor. For example, the SRSF (e.g., SRFS1) inhibitor is TG003, diospyrin, or chlorohexidine. In other aspects, the splicing inhibitor is selected from the group consisting of spliceostatins A-G, thailanstatins A-C, meamycins, Pladienolide B inhibitors (e.g., pladienolides A-G, E7107, or FD-895), Herboxidiene (GEX1A) inhibitors (e.g., GEX1Q1-5, RQN-18690A (18-deoxyherboxidiene)), Sudemycins (e.g., Cl, D1, F1, E, D6), Isoginkgetin, Madrasin (e.g., DDD00107587), Tetrocarcin A (e.g., NSC333856), N-palmitoyl-L-leucine, Psoromic acid, Clotrimazole, NSC635326, Napthazarin (e.g., NSC659999, BN82865, or NSC95397), Translation Inhibitors (e.g, Erythromycin, Cl-tetracycline, or treptomycin), HDAC inhibitors (e.g., SAHA, Splitomicin, or DHC), HAT inhibitors (e.g., Garcinol, AA, or BA3), PP1/PP2A inhibitors (e.g., Okadaic acid, Tautomycin or microcystin-LR), Ubistatin A, or G5 (Effenberger et al., 2016).

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is non-small cell lung cancer.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

A. Pharmaceutical Compositions

Also provided herein are pharmaceutical compositions and formulations comprising an NMD inhibitor and a pharmaceutically acceptable carrier for subjects determined to have a p53-deficient cancer.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve NMD inhibition in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

The NMD inhibitor may be administered before, during, after, or in various combinations relative to an additional cancer therapy, such as immune checkpoint therapy. The administrations may be in intervals ranging from concurrently to minutes to days to weeks. In embodiments where the NMD inhibitor is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below NMD inhibitor is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammaII and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-C SF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129; International Patent Publication Nos. WO 01/14424, WO 98/42752, and WO 00/37504 (CP675,206, also known as tremelimumab; formerly ticilimumab); U.S. Pat. No. 6,207,156; Hurwitz et al., 1998; Camacho et al., 2004; and Mokyr et al., 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application Nos. WO2001014424, and WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. KIT

Also within the scope of the present disclosure are kits for detecting p53-deficiency, such as those disclosed herein. An example of such a kit may include a set of p53 mutation-specific primer. The kit may further comprise instructions for use of the primers to detect the presence or absence of the specific p53 mutations described herein. The kit may further comprise instructions for diagnostic purposes, indicating that a positive identification of p53 mutations described herein in a sample from a cancer patient indicates sensitivity to the NMD inhibitor or a structurally similar inhibitor. The kit may further comprise instructions that indicate that a positive identification of p53 mutations described herein in a sample from a cancer patient indicates that a patient should be treated with a NMD inhibitor, or a structurally similar inhibitor.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Targeting Nonsense-Mediated Decay to Activate p53 Pathway

The NMD inhibitor, IACS14140 (compound 11), which selectively inhibits the phosphorylation of UPF1 by SMG1, a critical step for NMD, was used to investigate whether NMD inhibition triggers p53β/γ expression and subsequently activates the p53 pathway in MDM2 overexpressing cells bearing WT TP53. NSCLC (H460, H1944 and A549) and glioblastoma (GBM) (GSC289 and GSC231) cell lines were used. MDM2 was overexpressed in H1944 and A549 relative to H460 (FIG. 5A), and in GSC231 relative to GSC289 (FIG. 5B). These cell lines were treated with IACS 14140 (herein onwards referred to as NMDi) (1 μM) and using p53β and p53γ isoform specific primers, assessed their mRNA expression level (FIG. 1C, 1D). While NMDi treatment significantly increased p53β mRNA expression in all five cell lines, only MDM2 overexpressing cells showed p53γ mRNA upregulation (FIG. 1C, 1D, 6A). Western blotting revealed that NMDi treatment induced a truncated p53 protein similar in size to p53β/γ (FIG. 1E, 6B). These results indicate that NMD regulates the expression of both p53β and p53γ isoforms.

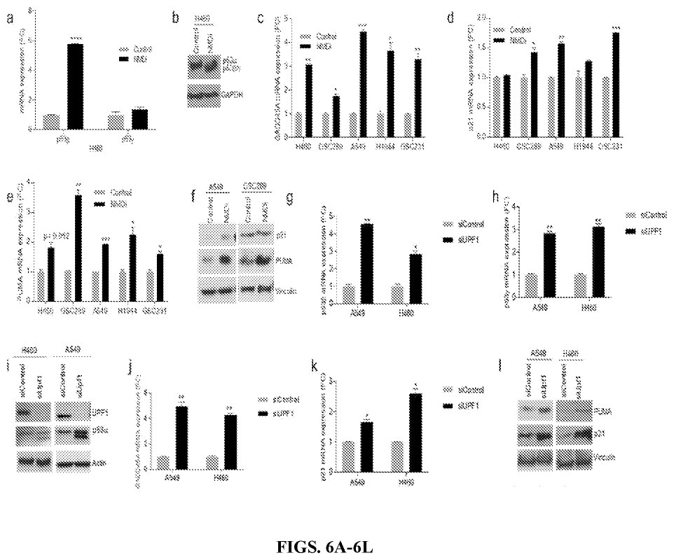

To examine whether NMD inhibition activates p53 pathway, the mRNA expression level of p53 transcriptional targets GADD45A, p21 and PUMA was measured and the results indicated an upregulation of these transcripts upon NMD inhibition (FIG. 6C, 6D, 6E). Western analysis showed an increased expression of PUMA protein in A549 and GSC289, and that of p21 in A549 (FIG. 6F). Assessment of p53β and p53γ mRNA expression in A549 and H460 cells depleted of UPF1, a key component of NMD pathway, further confirmed the NMD-susceptibility of these isoforms (FIG. 6G, 6H, 6I). UPF1-depletion also led to increased mRNA expression of GADD45A and P21 (FIG. 6J, 6K) and increased expression of p21 and PUMA proteins (FIG. 6L). Collectively, these results suggest that NMD inhibition upregulates p53β and p53γ isoforms, overcomes the negative regulation of p53 by MDM2 overexpression and promotes re-activation of the p53 pathway in cells bearing WT TP53.

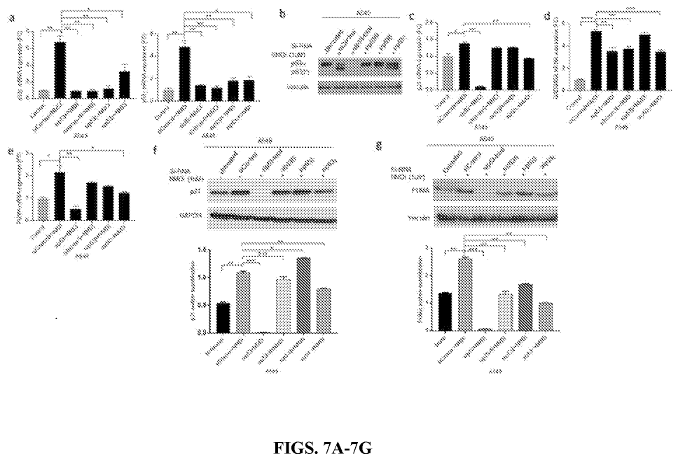

To further confirm that p53 isoforms induced by NMDi are indeed p53β and p53γ, and to determine which of these contribute to the enhanced p53 target gene expression and pathway activation, RNAi-mediated knockdown of total p53, intron-9, p53β and p53γ was performed in A549 (FIG. 7). siRNAs targeting total p53 and intron-9 caused significant reduction in the NMDi-induced expression of p53β and p53γ transcripts and led to either the absence or reduced expression of truncated p53 protein (FIG. 7A, 7B). P53β and p53γ specific siRNAs also either abolished or reduced their NMDi-induced expression (FIG. 7A, 7B), confirming their identity. siRNAs targeting p53β also reduced the expression of p53γ and vice versa suggesting that their expression may be co-dependent (FIG. 7A, 7B). siRNA against total p53 led to a significant reduction in p21, GADD45A and PUMA transcripts (FIG. 7C, 7D, 7E). Intriguingly, among the two isoforms, cells with p53γ knock-down showed the most reduction in the expression of p21, GADD45A and PUMA (FIG. 7C, 7D, 7E). Western analysis corroborated with the mRNA expression (FIG. 7F, 7G), providing further evidence that p53β/γ isoforms promote p53 pathway activation upon NMD inhibition and that the relative contribution of p53γ appears to be greater than that of p53β in these tumor cells.

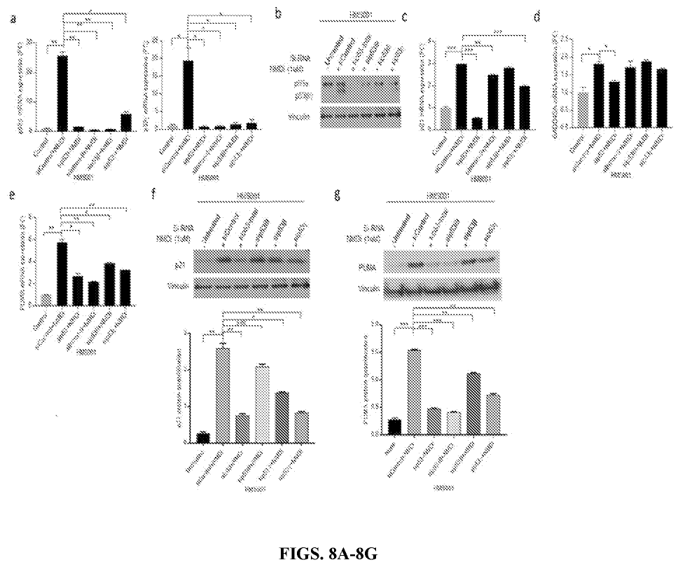

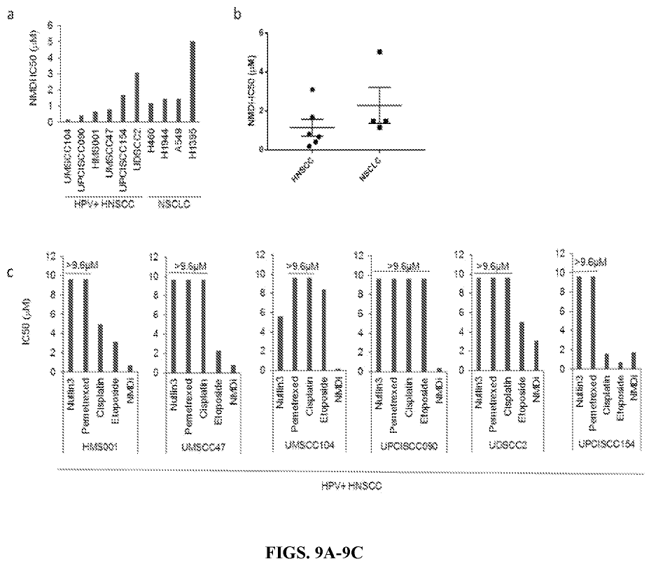

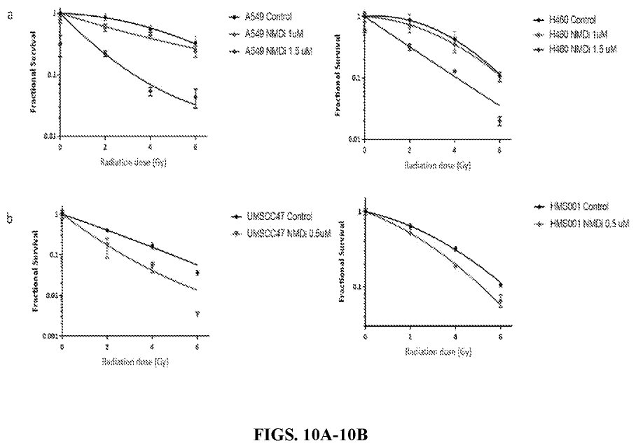

Given the findings that NMD inhibition induces p53β and p53γ, and promotes p53 pathway activation in MDM2-overexpressing cells (FIG. 1C, 1D, 1E, 6), it was examined whether targeting NMD induces similar response in HPV⁺ cells bearing WT p53 and hence serve as a potential therapeutic approach for these tumors. Six HPV⁺ head and neck squamous cell carcinoma (HNSCC) cell lines were used, two of which (HMS001 and UPCISCC090) also showed MDM2 overexpression (FIG. 1C). In these cells, NMDi treatment significantly increased mRNA levels of both p53β and p53γ (FIG. 1G, 1G) and induced the expression of the truncated p53 protein (FIG. 1H). RNAi-mediated knockdown in HMS001 further confirmed the identity of p53β and p53γ (FIG. 8A, 8B). NMDi significantly increased p21, GADD45A and PUMA mRNA expression (FIG. 1I, 1J, 1K) and PUMA and p21 protein expression, in at least five of the cell lines (FIG. 15M). Isoform-specific knockdown in HMS001 indicated that p53γ contributes to the increased expression of p53 transcriptional targets PUMA and p21 upon NMD inhibition (FIG. 4C, 4D, 4E). Western analyses supported this finding (FIG. 4F, 4G). These results indicate that NMD inhibition effectively reactivates the p53 pathway in HPV⁺HNSCC regardless of MDM2 expression status and that this reactivation is dependent at least in part on the p53γ isoform.

To test whether NMDi treatment rescues p53 function in mRNA transcripts bearing PTCs at the C-terminal exons, H2228, TCCSUP and UACC-893 cells were utilized, derived from non-small cell lung cancer (NSCLC), urinary bladder cancer and breast cancer respectively, bearing PTCs at p53 codons 331 (H2228), 342 (TCCSUP) and 349 (UAC-893) (FIG. 8A). NMDi treatment increased the expression of mutant p53 at both mRNA and protein levels (FIG. 2B, 2C). In addition, NMDi treatment also led to an increased expression of p53β/γ transcripts in these cells (FIG. 2D, 2E). Moreover, there was a significant increase in the mRNA expression of p53 transcriptional target genes P21, PUMA and GADD45A (FIG. 2F, 2G, 2H). The results also showed an increased expression of p21 protein in all three cell lines and that of PUMA in TCCSUP and UACC-893 cells (FIG. 21). These results demonstrate that NMDi protects PTC-bearing p53 transcripts from degradation and rescues p53 functions.

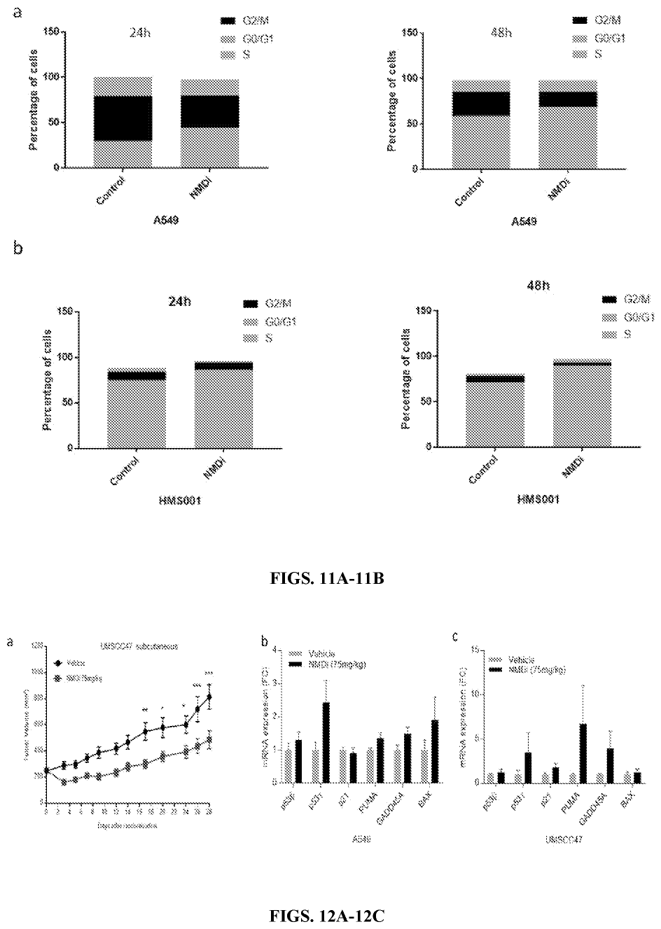

Increased expression of p53 target genes upon NMD inhibition prompted exploration of whether NSCLC and HPV⁺HNSCC cell lines are sensitive to NMDi and whether NMDi enhances radiation sensitivity. Cell viability assay demonstrated that both NSCLC and HPV⁺HNSCC cell lines are sensitive to NMDi (FIG. 9A). Interestingly, HPV⁺HNSCC cells were more sensitive to NMDi than MDM2-overexpressing NSCLC cells (FIG. 9B). NMDi was more potent compared to MDM2 inhibitor nutlin and commonly used chemotherapy agents, cisplatin, etoposide and pemetrexed (FIG. 10C). NMDi also increased the radiotherapy sensitivity of NSCLC and HPV⁺HNSCC cells (FIGS. 11A, 11B). Taken together, these results indicate that NMD inhibition enhances p53 pathway activation and subsequently inhibits tumor cell viability and sensitizes tumor cells to radiotherapy.

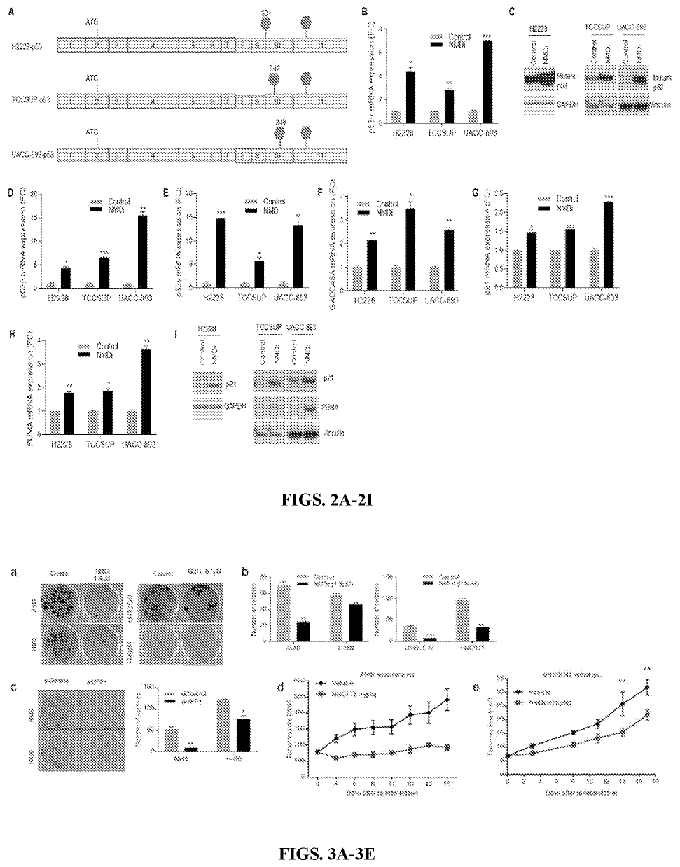

Because NMD inhibition upregulated p21, cell cycle analysis was performed. Results showed an increased G0/G1 and decreased G2/M populations in NMDi treated cells, indicating disruption in cell cycle progression (FIG. 11A, 11B). NMDi treatment also led to a significant reduction in the colony forming ability of NSCLC and HPV⁺HNSCC cells (FIG. 10A, 10B). UPF1-depletion in A549 and H460 cells showed similar results (FIG. 3C), further confirming the finding.

To test the therapeutic benefits of NMD inhibition in vivo, tumor growth of A549 and HPV⁺ UMSCC47 cells was assessed in an immune-deficient mouse model (FIG. 3D, 3E, 12A). NMDi treatment significantly impaired tumor growth in subcutaneous models of both A549 and UMSCC47 (FIG. 3D, 12A) and in an orthotopic model of UMSCC47 in which tumor cells were injected into the tongue (FIG. 3E). NMDi-treated A549 tumors exhibited increased p53γ and BAX mRNA expression and UMSCC47 tumors showed a higher expression of p53γ, p21, GADD45A and PUMA (FIG. 12B, 12C). Collectively, these results demonstrate that in addition to activating p53 pathway, NMD inhibition also suppresses tumor growth in NSCLC and HPV⁺HNSCC preclinical models.

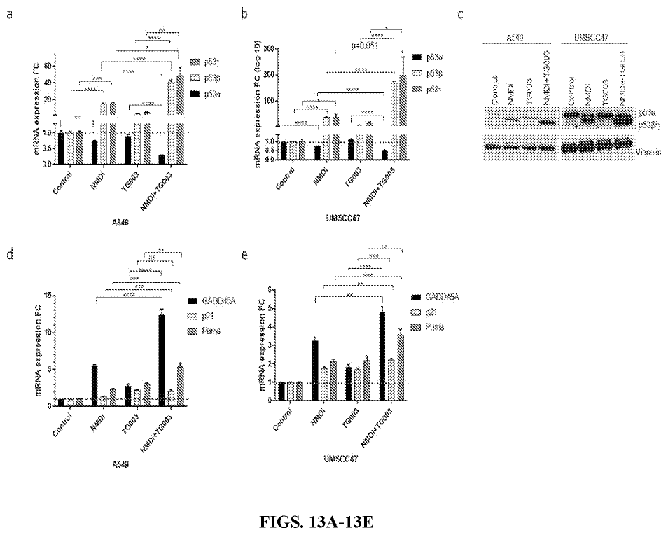

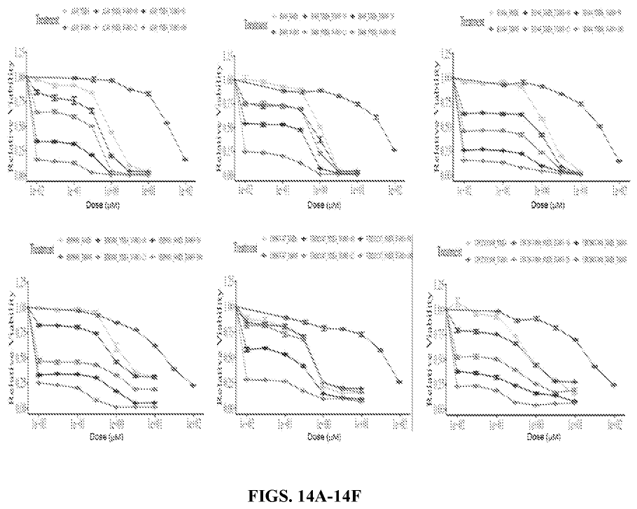

It was assessed whether NMD inhibition in conjunction with mRNA splicing perturbation of SRSFs further enhances the expression of p53β/γ isoforms, by treating A549 and UMSCC47 cell lines with a combination of NMDi and a Clk inhibitor, TG003. Decreased expression of p53α and increased expression of p53β/γ was observed indicating a splicing shift (FIG. 13A, 13B, 13C). Combination of NMDi and TG003 also led to further increases in the mRNA expression of p53 transcriptional targets (FIG. 13D, 13E). To test the therapeutic benefits, cell viability assay was performed. Results demonstrated that NSCLC and HNSCC cells displayed increased sensitivity to the combination of NMDi and TG003 treatment than either alone. (FIG. 14). These data provide evidence that the reactivation of the p53 pathway, and the potential therapeutic efficacy of NMD inhibition, could be further enhanced through combinations with agents that promote the alternative splicing of p53 to favor production of the p53β/γ isoforms over the full length p53γ isoform (FIG. 4).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., Current Protocols in Molecular Biology, John Wiley &amp; Sons, New York, N.Y., 2003.
Bukowski et al., Clinical Cancer Res., 4(10):2337-2347, 1998.
Camacho et al. J Clin Oncology 22(145): Abstract No. 2505 (antibody CP-675206), 2004.
Chee et al., Science, 274:610-614, 1996.
Cheruiyot et al., PLoS One, 13(10), 2018
Cho et al., Cancer Res 73:6770-9, 2013.
Christodoulides et al., Microbiology, 144(Pt 11):3027-3037, 1998.
Church and Gilbert, Proc. Natl. Acad. Sci. USA 81:1991-1995 (1988).
Cotton et al., Proc. Natl. Acad. Sci. USA 85:4397-4401 (1985).
Davidson et al., J Immunother., 21(5):389-398, 1998.
Del Tito et al., Clinical Chemistry 44:731-739, 1998.
Drmanac et al., Nat. Biotechnol., 16:54-58, 1998.
Drmanac et al., Science, 260:1649-1652, 1993.
Effenberger et al., Wiley Interdiscip Rev RNA, 8(2), 2017.
Flavell et al., Cell 15:25 (1978).
Fu et al., Nat. Biotechnol., 16:381-384, 1998.
Geever et al., Proc. Natl. Acad. Sci. USA 78:5081 (1981).
Gotham et al., Org. Biomol. Chem, 14, 1559-1563, 2015.
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Hurwitz et al. Proc Natl Acad Sci USA 95(17): 10067-10071, 1998.
International Patent Publication No. WO 99/57318
International Patent Publication No. WO1995001994
International Patent Publication No. WO1998042752
International Patent Publication No. WO2000037504
International Patent Publication No. WO2001014424
International Patent Publication No. WO2009/101611
International Patent Publication No. WO2009/114335
International Patent Publication No. WO2010/027827
International Patent Publication No. WO2011/066342
International Patent Publication No. WO2015016718
Leal, M., Ann NY Acad Sci 1321, 41-54, 2014.
Meric-Bernstam et al., Clin Cancer Res, 2018.
Mokyr et al. Cancer Res 58:5301-5304, 1998.
Pardoll, Nat Rev Cancer, 12(4): 252-64, 2012.
Qin et al., Proc. Natl. Acad. Sci. USA, 95(24):14411-14416, 1998.
Raca et al., Genet Test 8(4):387-94 (2004).
Sanger et al., Proc. Natl. Acad. Sci. USA 74:5463-5467 (1977).
Sears et al., Biotechniques, 13:626-633, 1992.
Sheffield et al., Proc. Natl. Acad. Sci. USA 86:232-236 (1989).
U.S. Pat. No. 4,870,287
U.S. Pat. No. 5,288,644
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,869,245
U.S. Pat. No. 5,885,796
U.S. Pat. No. 6,207,156
U.S. Pat. No. 8,008,449
U.S. Pat. No. 8,017,114
U.S. Pat. No. 8,119,129
U.S. Pat. No. 8,329,867
U.S. Pat. No. 8,354,509
U.S. Pat. No. 8,735,553
U.S. Patent Publication No. 2004/0014095
U.S. Patent Publication No. 2005/0260186
U.S. Patent Publication No. 2006/0104968
U.S. Patent Publication No. 20110008369
U.S. Patent Publication No. 2014022021
U.S. Patent Publication No. 20140294898
Underhill et al., Genome Res. 7:996-1005 (1997).
Zimmerman et al., Methods Mol. Cell. Biol., 3:39-42, 1992.

What is claimed is:

1. A method of treating a subject determined to have a p53-deficient cancer, the method comprising restoring p53 function in the subject's tumor by administering a therapeutically effective amount of an inhibitor of nonsense-mediated decay (NMD) to said subject.

2. The method of claim 1, wherein the p53-deficient cancer comprises a C-terminal p53 mutation.

3. The method of claim 2, wherein the C-terminal p53 mutation is a nonsense mutation or missense downstream of exon 9.

4. The method of any of claims 1-3, wherein the p53-deficient cancer comprises mutant p53 with premature termination codons (PTCs) in the coding region.

5. The method of claim 4, wherein the PTCs are at residue 331, 342, or 349.

6. The method of any of claims 1-5, wherein the p53-deficient cancer comprises the p53α or p53β isoform The method of any of claims 1-6, wherein the p53-deficient cancer comprises the p53γ isoform.

8. The method of any of claims 1-7, wherein the p53-deficient cancer comprises the p53γ isoform.

9. The method of any of claims 1-8, wherein the p53-deficient cancer is a MDM2 amplified or overexpressing cancer.

10. The method of any of claims 1-9, wherein the p53-deficient cancer is a MDMX amplified or overexpressing cancer.

11. The method of any of claims 1-11, wherein the p53-deficient cancer is an HPV-positive cancer.

12. The method of any of claims 1-11, wherein the subject was determined to have a p53 mutation by analyzing a genomic sample from said subject

13. The method of claim 12, wherein the genomic sample is isolated from saliva, blood, urine, normal tissue, or tumor tissue.

14. The method of claim 12 or 13, wherein the presence of a p53 mutation is determined by nucleic acid sequencing or PCR analyses.

15. The method of any of claims 1-14, wherein the inhibitor of NMD is 5-azacytidine, Caffiene, wortmannin, NMDI-1, NMDI14 (530838), VG1, Compound C, Proscillaridin, Lanatoside C, ouabain, Cycloheximide, Pateamine A (PatA), Emetine, Digitoxin, digoxin, doxorubicin, IACS 14140, Ataluren (Translarna), Docusate sodium, butyl paraben, ethacridine lactate, Thapsigargin, Calcium ionophore A23187, Curcumin, Geneticin, Gentamicin, Negamycin, RTC #13, RTC #14, G-418, PTC124 (Ataluren), Tylosin or Amlexanox.

16. The method of any of claims 1-16, wherein the inhibitor of NMD is antisense RNA or siRNA targeted to RENT1, RENT2, eIF4A, UPF1, UPF2, UPF3B, RNPS1, Y14, MAGOH, NMD1 or SMG.

17. The method of any of claims 1-17, further comprising determining whether p53 function is restored by measuring an increased mRNA expression level of p53 transcriptional targets.

18. The method of claim 17, wherein p53 targets are MDM2, MDMX, GADD45A, p21, PUMA, GDF15, SUSD6 (TMPS, DRAGO, KIAA0247), GADD45A, PLK3, BTG2, TIGAR (C12orf5), TNFRSF10B, PPM1D, AEN, PLK2, SESN1, FAS and/or KITLG.

19. The method of any of claims 1-18, further comprising administering a therapeutically effective amount of an inhibitor of mRNA splicing.

20. The method of claim 19, wherein the inhibitor of mRNA splicing is a serine/arginine-rich splicing factor (SRFS) inhibitor.

21. The method of claim 20, wherein the inhibitor of mRNA splicing is a serine/arginine-rich splicing factor 1 (SRFS1, ASF1/SF2) inhibitor.

22. The method of claim 20, wherein the SRFS inhibitor is TG003, diospyrin, a SF3B1 inhibitor, a thailanstatin, chlorohexidine, SPHINX 31, or SRPIN 340.

23. The method of any of claims 1-23, further comprising administering to the subject an additional anti-cancer therapy.

24. The method of claim 23, wherein the additional anti-cancer therapy is chemotherapy, radiotherapy, gene therapy, surgery, hormonal therapy, anti-angiogenic therapy or immunotherapy.

25. The method of claim 23, wherein the additional anti-cancer therapy is radiotherapy, chemotherapy, radiation, immunotherapy, or a combination thereof.

26. The method of any of claims 23-25, wherein the additional anti-cancer therapy is an MDM2 targeting drug,

27. The method of claim 26, wherein the MDM2 targeting drug is Nutlin-3, RG7112 (RO5045337), RG7388 (RO5503781, Idasanutlin), SAR405838 (MI-77301), MK-8242 (SCH 900242), DS-3032b, HDM201, CGM097, ALRN-6924 or AMG-232.

28. The method of any of claims 1-27, wherein the NMD inhibitor and/or additional anti-cancer therapy are administered intravenously, subcutaneously, intraosseously, orally, transdermally, in sustained release, in controlled release, in delayed release, as a suppository, or sublingually.

29. The method of any of claims 1-28, wherein administering the NMD inhibitor and/or anti-cancer therapy comprises local, regional or systemic administration.

30. The method of any of claims 1-29, wherein the NMD inhibitor and/or anti-cancer therapy are administered two or more times.

31. The method of any of claims 1-30, wherein the subject is human

32. The method of any of claims 1-31, wherein the cancer is sarcoma, breast cancer, lung cancer, or glioblastoma.

33. The method of claim 32, wherein the lung cancer is non-small cell lung cancer.

34. The method of claim 32, wherein the lung cancer is small cell lung cancer.

35. The method of claim 32, wherein the cancer is a glioblastoma.

36. The method of any of claims 1-35, wherein the cancer is head and neck squamous cell carcinoma, oropharyngeal cancer, cervical cancer, anal cancer, penile cancer, or vulvar cancer.

37. A pharmaceutical composition comprising an NMD inhibitor and mRNA splicing inhibitor.

38. The composition of claim 37, wherein the NMD inhibitor is 5-azacytidine, Caffiene, wortmannin, NMDI-1, NMDI14 (530838), VG1, Compound C, Proscillaridin, Lanatoside C, ouabain, Cycloheximide, Pateamine A (PatA), Emetine, Digitoxin, digoxin, doxorubicin, IACS 14140, Ataluren (Translarna), Docusate sodium, butyl paraben, ethacridine lactate, Thapsigargin, Calcium ionophore A23187, Curcumin, Geneticin, Gentamicin, Negamycin, RTC #13, RTC #14, G-418, PTC124 (Ataluren), Tylosin or Amlexanox.

39. The composition of claim 37 or 38, wherein the inhibitor of NMD is antisense RNA or siRNA targeted to RENT1, RENT2, eIF4A, UPF1, UPF2, UPF3B, RNPS1, Y14, MAGOH, NMD1, SMG or UPF1.

40. The composition of any of claims 37-39, wherein the mRNA splicing inhibitor is TG003, diospyrin, a SF3B1 inhibitor, a thailanstatin, chlorohexidine, SPHINX 31, or SRPIN 340.

41. A composition comprising an effective amount of an NMD inhibitor and mRNA splicing inhibitor for use in the treatment of a p53-deficient cancer.

42. The use of composition of claim 37 for the treatment of a p53-deficient cancer in a subject.

43. A method of identifying a subject who is a candidate for an NMD inhibitor therapy comprising analyzing the sequence of a p53 gene from a cancer in the subject having been determined to have a p53-deficient cancer.

44. The method of claim 43, wherein the cancer is an HPV-positive cancer.

45. The method of claim 43 or 44, wherein the sequence is analyzed from a genomic sample from said subject.

46. The method of any of claims 43-45, wherein the genomic sample is isolated from saliva, blood, urine, normal tissue, or tumor tissue.

47. The method of any of claims 43-46, wherein analyzing comprises performing nucleic acid sequencing or PCR analyses.

48. The method of any of claims 43-47, wherein a subject determined to have a p53 nonsense mutation downstream of exon 9 and/or PTCs in the p53 coding region is a candidate for an NMD inhibitor therapy.

49. The method of claim 48, wherein the PTCs are at residue 331, 342, or 349.

50. The method of any of claims 43-49, wherein a subject determined to have a p53β isoform or p53γ isoform is a candidate for an NMD inhibitor therapy.

51. The method of any of claims 43-50, further comprising administering an NMD inhibitor therapy to said subject identified to be a candidate.

52. The method of any of claims 43-51, wherein the NMD inhibitor therapy comprises administering 5-azacytidine, Caffiene, wortmannin, NMDI-1, NMDI14 (530838), VG1, Compound C, Proscillaridin, Lanatoside C, ouabain, Cycloheximide, Pateamine A (PatA), Emetine, Digitoxin, digoxin, doxorubicin, IACS 14140, Ataluren (Translarna), Docusate sodium, butyl paraben, ethacridine lactate, Thapsigargin, Calcium ionophore A23187, Curcumin, Geneticin, Gentamicin, Negamycin, RTC #13, RTC #14, G-418, PTC124 (Ataluren), Tylosin or Amlexanox.

53. The method of any of claims 43-53, further comprising administering a therapeutically effective amount of an inhibitor of mRNA splicing.

54. The method of claim 53, wherein the inhibitor of mRNA splicing is a serine/arginine-rich splicing factor (SRFS) inhibitor.

55. The method of claim 53, wherein the inhibitor of mRNA splicing is a serine/arginine-rich splicing factor 1 (SRFS1, ASF1/SF2) inhibitor.

56. The method of claim 55, wherein the SRFS inhibitor is TG003, diospyrin, a SF3B1 inhibitor, a thailanstatin, chlorohexidine, SPHINX 31, or SRPIN 340.

57. The method of any of claims 43-56, wherein the NMD inhibitor therapy comprises administering an antisense RNA or siRNA targeted to RENT1, RENT2, eIF4A, UPF1, UPF2, UPF3B, RNPS1, Y14, MAGOH, NMD1, SMG or UPF1.
